(19) United States
(12) Patent Application Publication
(10) Pub. No.: US 2014/0301866 A1
(43) Pub. Date: Oct. 9, 2014

(54) DEVICE FOR DETECTING AND MEASURING THE ROTATION OF A PERISTALTIC CASSETTE

(71) Applicant: Frédéric Neftel, Lausanne (CH)

(72) Inventor: Frédéric Neftel, Lausanne (CH)

(73) Assignee: BRACCO INJENEERING SA, Lausanne (CH)

(21) Appl. No.: 14/006,046

(22) PCT Filed: Mar. 15, 2012
(86) PCT No.: PCT/IB12/51246

§ 371 Date: Jan. 29, 2014

(30) Foreign Application Priority Data
Mar. 21, 2011 (EP) 11159081.6

(51) Int. Cl. F04B 51/00; A61M 5/142; A61M 5/168; F04B 43/12

(52) U.S. Cl. CPC F04B 51/00; F04B 43/1261; A61M 5/14232; A61M 5/16831
USPC 417/53; 417/477.9; 417/63

(57) ABSTRACT

The invention relates to a device for detecting and identifying a peristaltic cassette which is provided with a rotary mechanism comprising rollers connected to a spacer (1) and which is intended to be rotated by a drive system connected to the device. Said device comprises a sensor arranged such as to detect the passage of at least one distinctive element (2) disposed on the spacer, by counting the number of distinctive elements detected during one cassette rotation.

### FIELD OF THE INVENTION

The present invention relates to peristaltic cassettes comprising a roller unit that is rotated about an axis. The invention is particularly intended for the medical field and its purpose is to ensure that said cassette is recognised by the drive system to prevent counterfeiting thereof or problems with the operation and/or characteristics of the cassette being used.

### STATE OF THE ART

An example of a cassette corresponding to the definition in the preceding section is disclosed in patent application WO 2005/106251, and in the Malbec patent EP0388269.

The cassette comprises a series of rollers that are rotated about a central motor shaft, wherein each roller has an axis of which at least one end is mounted slidably in a radial direction in the slot of a plate called a “spacer” that is disposed on one side of the cassette, wherein said plate itself is fixed so as to be immovable relative to motor shaft and is thus driven by the rotation of the rollers, which in turn are driven by friction with the central shaft, for example.

The rotating speed of the rollers must be determined with the highest possible degree of accuracy because this enables the flow velocity or flow rate of the liquid to be determined as it passes through the cassette.

### GENERAL DESCRIPTION OF THE INVENTION

The problem the present invention is designed to solve consists in improving the detection and measurement of the rotation of a peristaltic cassette and/or the identification of a cassette.

In the invention, the solution to the above problem has the form of a device for detecting and identifying a peristaltic cassette furnished with a rotating mechanism that comprises rollers connected to a spacer, wherein said cassette is designed to be rotatable by a drive system connected to the device, said device being characterized in that it comprises a sensor fixed immovably to the drive system arranged to detect the passage of at least one distinctive element disposed on said spacer by counting the number of distinctive elements detected during one rotation of the cassette.

In principle, the spacer is defined by two essentially opposite surfaces, an inner surface which faces the inside of the cassette and an outer surface which faces the outside of the cassette. In this case, the device is characterized in that it comprises a sensor arranged so as to detect the passage of at least one distinctive element visible on said outer surface. The spacer may also be connected to the rollers or to any other means for ensuring rotation that is proportional with the rotation of the rollers.

The distinctive elements are preferably located on a ring that is moved into position opposite said sensor during the rotation of the cassette. Said distinctive elements do not necessarily have any other function and in this case are superimposed on the functional elements of the cassette for the sole purpose of ensuring said detection or said identification of the cassette.

In one embodiment of the invention, the sensor is optical. In this case the spacer is furnished either with apertures that can be detected by the optical sensor as the spacer rotates, or with regions that are optically reflective or opaque (marked surfaces) that make it possible to ensure detection of said surfaces as they pass opposite the sensor, and thereby to identify the cassette.

The apertures or marked surfaces may be arranged symmetrically or asymmetrically on a diameter of the spacer located opposite the optical sensor and thus coded information that particularly enables the type of cassette used to be identified uniquely. In order to be able to read the information carried by the spacer in this way, the cassette must be rotated through at least one revolution.

Identification of this type may be particularly useful when the intention is to prevent the use of counterfeit cassettes for medical use, so that only cassettes guaranteed by the original manufacturer can be used by the driving device.

One example is that of contrast product injectors, which require a high degree of operating accuracy in order to ensure that injection in the patient is not interrupted or altered. A malfunction during injection would represent a significant risk of having to repeat the CT scanner examination and thus also a risk of having to irradiate the patient with x-rays, sometimes at a substantial level, a second time. The use of such a mechanism enabling identification of an original cassette (for example from the nature of the sequence of apertures or marked surfaces recognised as the cassette rotates) makes it possible to prevent the use of a cassette that has not been produced by an original manufacturer. Such cassette marking is located inside the cassette and requires that the cassette to be rotated be operated inside the appliance (the injector for example) for detection to take place. The user is thus unable to falsify the detection mode for example by adding a marking or label to the outside of the cassette, or by opening the cassette, which must remain sterile and would possibly be destroyed by such an operation.

Moreover, by detecting the rotation of the cassette during injection it is possible to verify proper operation of the cassette as well as the quantity actually injected over time by counting the number of rotations performed by the cassette and comparing this with the expected number of rotations based on the true number of rotations of the drive shaft.

Such a cassette marking system also serves to inform the injector of the type of cassette used, the performance and/or the expiration date thereof, to prevent an outdated cassette from being used. In this way, it is possible to impart the specific desired information via the sequence of markings and the spacing thereof.

An example of use of the invention in the context of contrast agent injectors for CT scanner imaging, such as the 3D and 4D CT Express marketed by the applicant, is the detection and recognition of the distinction between cassettes designed for a single patient (CT Express 3D) and cassettes that enable use for multiple patients (CT Express 4D), the latter being intended required for prolonged use, for as much as 4 injected litres compared with 300 ml for other cassettes.

Of course, the invention is not limited solely to the embodiments described above and can be used advantageously in any kind of device that requires identification of a type of rotary system for fluid administration while ensuring high reliability against counterfeiting of any kind.

### DETAILED DESCRIPTION OF THE INVENTION

The invention is described in greater detail in the following using examples illustrated by the following figures:

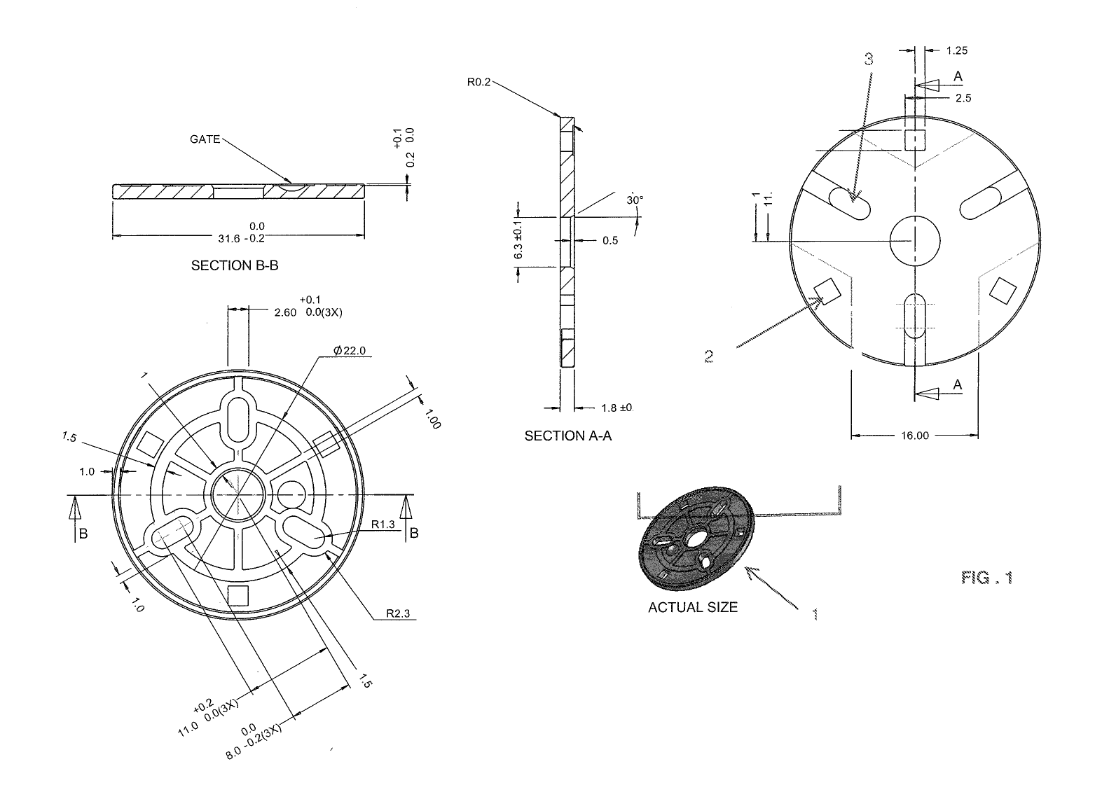

FIG. 1 illustrates various cross sections and views of a spacer according to the invention.

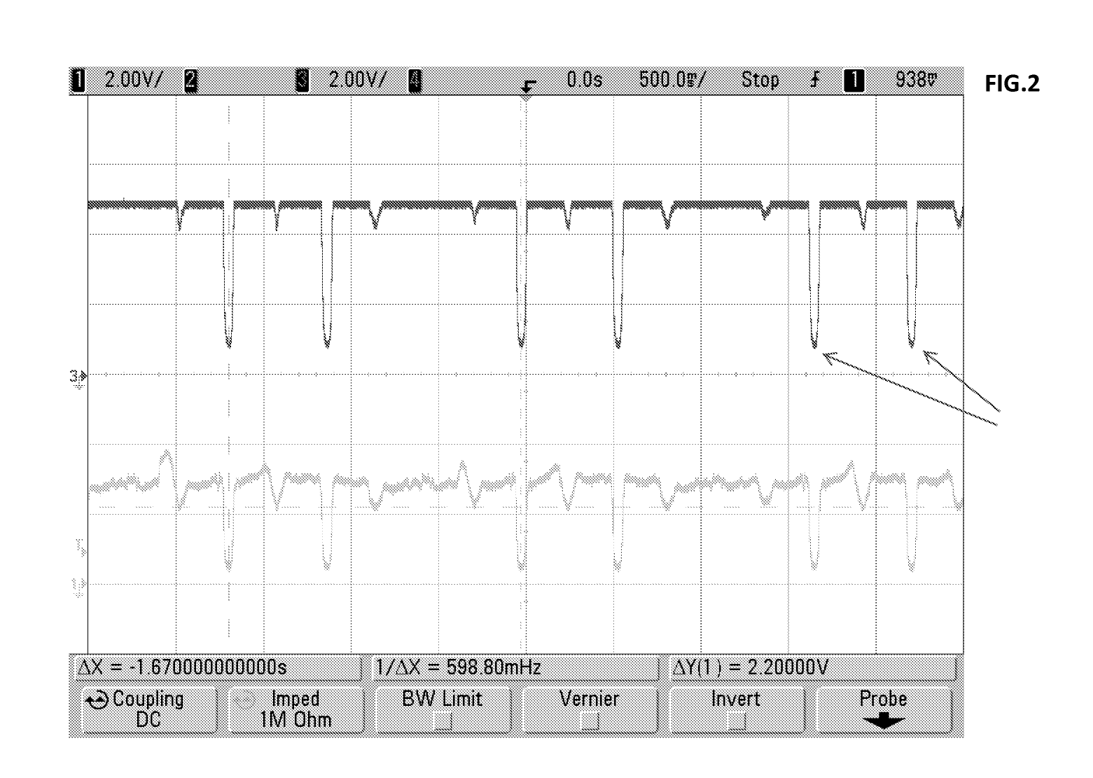

FIG. 2 shows an oscilloscope screenshot showing the signal recorded by an optical sensor.

FIG. 1 shows a spacer 1 having a shape quite similar to the one shown in application WO 2005/106251 but differing in that it has three apertures 2 arranged on a ring, the diameter of which is greater than another ring in which slits 3 are arranged for guiding the rollers.

In this example, the three apertures 2 have square cross sections. Any other cross-sectional shape or number of apertures can be used.

The oscilloscope curve in FIG. 2 shows valleys (indicated by arrows) that show the passing of the apertures in front of the optical sensor. The general shape of this curve thus enables the type of cassette (since each type has a specific number of ports) and its authenticity easily and quickly.

1. A device for the detection and identification of a peristaltic cassette furnished with a rotating mechanism comprising rollers connected to a spacer and designed to be rotated by a drive system connected to the device; said device being characterized in that it comprises a sensor arranged in such manner as to detect the passage of at least one distinctive element disposed on said spacer, by counting the number of distinctive elements detected during rotation of the cassette.

2. The device according to claim 1, wherein the spacer is essentially defined by two opposite surfaces, an inner surface facing the inside of the cassette and an outer surface facing outwardly from the cassette, the distinctive element being detectable at least on said external surface.

3. The device according to claim 1, wherein the sensor is an optical sensor.

4. The device according to claim 1, wherein the sensor is a Hall effect sensor and the distinctive element is a magnet.

5. A cassette designed to be used with a device according to claim 1, wherein the distinctive element is a reflective surface.

6. A cassette designed to be used with a device according to claim 1, wherein the distinctive element is an aperture.

7. The cassette according to claim 6 comprising a plurality of distinctive elements in the form of apertures.

8. The cassette according to claim 5, wherein at least one aperture or reflective surface has a different length than the other apertures in the axis for reading in rotation by the optical sensor.

9. The cassette according to claim 8, wherein the orifices or reflective surfaces are arranged at irregular intervals.

10. The cassette according to claim 8, wherein the intervals represent an identification sequence specific to the cassette.

11. The cassette according to claim 6, wherein the one or more distinctive elements are arranged on a circular trajectory of the spacer.

12. A unit consisting of a device and a cassette as defined in claim 1.

13. A method for determining the rotating speed of a cassette as defined in claim 1, comprising a step in which the number of distinctive elements detected by a sensor during a predefined time period is calculated, said speed being compared with the expected speed based on the driving speed of the cassette spindle.

14. A method for differentiating between peristaltic cassettes intended for single and multiple use, the cassettes being such as are defined in claim 1, wherein the two types of cassettes are differentiated by the number of distinctive elements arranged on the outside surface of the spacer; said method consisting of differentiating between the two types of cassette by counting the number of distinctive elements detected in the course of during at least one complete rotation of the cassette.

15. The method according to claim 14 for differentiating between peristaltic cassettes intended for single and multiple use, wherein the cassette is driven at low speed when it is first inserted in the drive system in order to read the information borne on the spacer by means of the one or more distinctive elements by counting the number of distinctive elements detected during at least one complete rotation of the cassette and thereby determining the type thereof before any fluid injection programming.

16. A method for recognizing an original peristaltic cassette, wherein the cassette is driven at low speed when it is first inserted in the drive system in order to read the information borne on the spacer by means of the one or more distinctive elements by counting the number of distinctive elements detected during at least one complete rotation of the cassette and determining the authenticity thereof by comparing it to a reference sequence before any fluid injection programming.

17. Use of a detection device as defined in claim 1 to ensure the proper operation of a peristaltic cassette during the injection of a fluid.

18. Use of a detection device as defined in claim 1 for detection of a peristaltic cassette, said use comprising a step in which a measurement is carried out for at least one complete rotation of the cassette.
